(19) United States
(12) Patent Application Publication
(10) Pub. No.: US 2022/0180155 A1
(43) Pub. Date: Jun. 9, 2022

(54) SYSTEM AND METHOD THAT CAN LEARN TO RECOGNIZE AND PREDICT ERRORS IN THE COMPUTATIONAL DETECTION OF CELLS WITHIN TISSUES BASED ON THEIR GEOMETRIC AND MORPHOLOGICAL PROPERTIES AND PERFORM TISSUE, CELL AND SUBCELLULAR-SCALE DYNAMICS ANALYSIS WITHIN A SINGLE USER INTERFACE

(71) Applicant: Veena Chatti, Eureka, CA (US)

(72) Inventor: Veena Chatti, Eureka, CA (US)

(21) Appl. No.: 17/592,883

(22) Filed: Feb. 4, 2022

(51) Int. Cl. G06N 3/04; G06N 3/08; G16H 30/40

(52) U.S. Cl. CPC G06N 3/0481; G16H 30/40; G06N 3/08

(57) ABSTRACT

Exemplary embodiments of a system and method that can learn to predict and recognize errors in the computational detection of cells within tissues from one or more images based on one or more input measurements and display them on a graphical user interface alongside tissue-, cell- and subcellular-scale dynamics data, comprising: a computing device comprising a computational cell detection module configured to take input data and detect cells within tissues and calculate the cells' geometric and morphological properties, the computational cell detection module configured to implement a single-layered neural network to recognize erroneously detected cells within tissues, an error recognition module comprising sigmoid configured to take a weighted sum of measurements of the geometric and morphological properties and return a value between 0 and 1, computational cell detection module configured to recognize errors such as under-detection errors within tissues and record and feed errors to a database.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### TECHNICAL FIELD

The disclosed subject matter relates generally to systems and computational methods for automated image recognition and analysis. Particularly, the disclosure relates to a system and method that can learn to accurately recognize and predict errors in the computational detection of cells within tissues and display them on a graphical user interface (GUI) alongside tissue-, cell- and subcellular-scale dynamics data based on input measurements of specific geometric and morphological properties of the cells.

### BACKGROUND

Technological advances in fluorescence microscopy have enabled life scientists to gather vast amounts of data about cell- and tissue-scale dynamics of living tissues by imaging living cells within the context of their living tissue environments, and also to gather information about the subcellular-scale dynamics by experimentation involving photobleaching fluorescently labelled cells and monitoring fluorescence recovery. Despite these technological advances, methods and systems that enable life scientists to computationally bridge these three organizational scales in complex living tissues and make measurements of biological processes simultaneously across the scales within a single platform are rare. One reason for this is that at the cell and tissue organizational scales, accurately detecting and tracking cells in these datasets of complex living tissues remains a computational challenge. Prior attempts have implemented strategies involving feature extraction, template-matching, and statistical and/or algorithmic techniques. Cell-detection algorithms used by existing image analysis toolkits rely on edge-detection algorithms such as the watershed algorithm, described, for example, in Aiguoy et al, 2016, and Tek, 2008.

A list of cited references below:
1. Aigouy B., Umetsu D., Eaton S. (2016) Segmentation and Quantitative Analysis of Epithelial Tissues. In: Dahmann C. (eds) Drosophila. Methods in Molecular Biology, vol 1478. Humana Press, New York, N.Y.
2. Tek, 2008, Local Watershed Operators for Image Segmentation, U.S. Pat. No. 7,327,880 B2, United States Patent and Trademark Office.

By measuring the intensity of fluorescence markers at cell membranes, the watershed algorithm can be implemented to detect cell membranes (edges) as regions of high intensity, assign values to each pixel, and then assign a label to each detected watershed region (below threshold intensity) marking it as a cell, as seen in FIG. 1A. This implementation of the watershed algorithm has enabled cell and tissue biologists to computationally identify and track individual cells within living tissues, using time-lapse datasets of fluorescently-labeled cell membranes imaged with fluorescence microscopy. Information about the geometric and morphological properties of cells can be calculated by this implementation of the watershed algorithm. However, this implementation of the watershed algorithm tends to be error-prone; it may under- or over-detect cells, making both error types simultaneously, even in uniformly-labeled datasets.

Manually recognizing and recording errors are tedious and laborious tasks that waste precious analytical time, in which scientists spend hours scouring datasets to search for computational errors in cell detection amid thousands of observations. This stalls the next cell-tracking step, which cannot be performed until cell detection errors are recognized and corrected. Existing cell-detection algorithms tend not to incorporate computationally automated steps to recognize or predict errors in cell detection.

The following disclosure addresses this computational challenge by describing a system and method that can learn to recognize errors in the computational detection of cells within tissues based on their geometric and morphological properties. This system and method can be implemented into existing cell-detection algorithms and analytical toolkits to facilitate the automation of the prediction and recognition of errors made by these algorithms.

### SUMMARY

A simplified summary of the disclosure follows below. This summary is not an extensive overview of the disclosure and it does not identify key/critical elements of the system and method, nor does it delineate the scope of the system and method. Its purpose is to present some concepts disclosed herein in a simplified form as a prelude to the more detailed description that is presented later.

An objective of the present disclosure is directed towards a system that improves the computational cell detection process through its highly precise error-recognition and prediction.

Another objective of the present disclosure is directed towards a system that automates the error-recognition process and facilitates correction of cell-detection errors in a more reproducible and consistent manner.

Another objective of the present disclosure is directed towards a system that can learn to recognize and predict errors in cell detection within tissues based on input measurements of the geometric and morphological properties of cells.

Another objective of the present disclosure is directed towards a system that predicts and recognizes errors in datasets.

Another objective of the present disclosure is directed towards a system that receives an input of the weighted sum of measurements of the geometric and morphological properties of cells and returns a value between 0 and 1, representing an output and prediction of the model.

Another objective of the present disclosure is directed towards the system that acquires data from images of individual cells that are adhered to each other within tissues.

Another objective of the present disclosure is directed towards a diagnostic system that can learn to recognize abnormal cell morphologies in shape and size in cases where cells manifest statistically separable morphological and geometric and morphological properties when compared to healthy cells.

Another objective of the present disclosure is directed towards a system that achieves highly accurate results in error recognition and reduces overall computational time as well as the number of steps involved in computation.

Another objective of the present disclosure is directed towards a system that specifically addresses the under-detection of cells imaged within tissues.

Another objective of the present disclosure is directed towards a system that is implemented in a GUI to flag erroneously-detected cells with a red asterisk displayed on the centroid in every analyzed frame of an overlay of an output of watershed algorithm-based detection on an original dataset.

In an embodiment of the present disclosure, a computing device comprising a computational cell detection module configured to take the one or more input measurements and computationally identify one or more cells within tissues and calculate information about one or more geometric and morphological properties of the one or more cells on the computing device, the one or more input measurements comprising an area measurement, a perimeter measurement, an orientation measurement, and an equivalent diameter measurement.

In another embodiment of the present disclosure, the computational cell detection module configured to implement a single-layered neural network for recognition of a plurality of abnormal and/or erroneously-detected cell shapes within tissues.

In another embodiment of the present disclosure, the computational cell detection module comprising sigmoid configured to take a weighted sum of measurements of the one or more geometric and morphological properties and return a value between 0 and 1.

In another embodiment of the present disclosure, the computational cell detection module configured to output a number closer to 1 when an error is predicted and output a number closer to 0 when no error is predicted.

In another embodiment of the present disclosure, the computational cell detection module configured to recognize one or more errors such as under-detection errors within the tissues, display them on a GUI alongside tissue-, cell- and subcellular-scale dynamics data, and record the one or more errors and feed the one or more errors to a database, the computational cell detection module configured to generate an output analysis on the computing device, the output analysis comprising statically separable geometric and morphological properties of detected cells.

### BRIEF DESCRIPTION OF THE DRAWINGS

The system and method are more fully appreciated in connection with the following detailed description. Other objects and advantages of the system and method will become apparent to those skilled in the art upon reading the following detailed description of the preferred embodiments, in conjunction with the accompanying drawings, wherein like reference numerals have been used to designate like elements. However, for clear illustration, reference numerals of the same element in different figures might be omitted.

FIG. 1B is an observation image depicting an error type, in accordance with one or more exemplary embodiments.

FIG. 1C is another observation image depicting graphical representation of sigmoid (x) and sigmoid′ (x) of an activation function, in accordance with one or more exemplary embodiments.

FIG. 2A is a block diagram depicting a schematic representation of a system for error recognition in the computational detection of cells within tissues from images based on input measurements about the geometric and morphological properties of the cells, in accordance with one or more exemplary embodiments.

FIG. 2B is an example diagram depicting the computational cell detection module 208 shown in FIG. 2A, in accordance with one or more exemplary embodiments.

FIG. 3 is an example diagram depicting an information flow in the learning process by which the computational cell detection module learns to recognize and predict errors, in accordance with one or more exemplary embodiments.

FIG. 4 is an example diagram depicting diagrammatic representation of input measurements, in accordance with one or more exemplary embodiments.

FIG. 5 is an example diagram depicting frequency of occurrence of under detection errors in an example dataset, in accordance with one or more exemplary embodiments.

FIG. 6 is an example diagram depicting a process by which information is collected, curated for ground truth, and fed to the computational cell detection module 208 to learn, in accordance with one or more exemplary embodiments.

FIG. 7 is an example graph depicting statistically separable geometric and morphological properties of cells, in accordance with one or more exemplary embodiments.

FIG. 8 is an example graph depicting a 3-axis graphical representation of the effect of updating weight w and bias b on the value of cost, c, of prediction, p, in accordance with one or more exemplary embodiments.

FIG. 9 is an example graph depicting a matrix of faceted graphs with each vertical panel showing the effect of varying learning rate, and each horizontal panel showing the effect of varying the number of epochs used by the computational cell detection module 208 to learn, in accordance with one or more exemplary embodiments.

FIG. 10 is an example diagram depicting a test of the performance of the model of the computational cell detection module 208 over a dataset, in accordance with one or more exemplary embodiments.

FIG. 11 is a flowchart depicting a method for error recognition in the computational detection of cells within tissues from images based on input measurements of certain geometric and morphological properties of cells or certain combinations of certain geometric and morphological properties of cells, in accordance with one or more exemplary embodiments.

FIG. 12 is a flowchart depicting a method for recognizing erroneous cells using a GUI, in accordance with one or more exemplary embodiments.

FIG. 13 is an example diagram depicting a GUI, in accordance with one or more exemplary embodiments.

FIG. 14 is a flowchart depicting a method for displaying watershed-based cell detection and cell- and tissue-scale dynamics analyses alongside the analysis of the subcellular-scale dynamics results by using the GUI, in accordance with one or more exemplary embodiments.

FIG. 15 is a block diagram illustrating the details of a digital processing system in which various aspects of the present disclosure are operative by execution of appropriate software instructions.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and so forth, herein do not denote any order, quantity, or importance, but rather are used to distinguish one element from another.

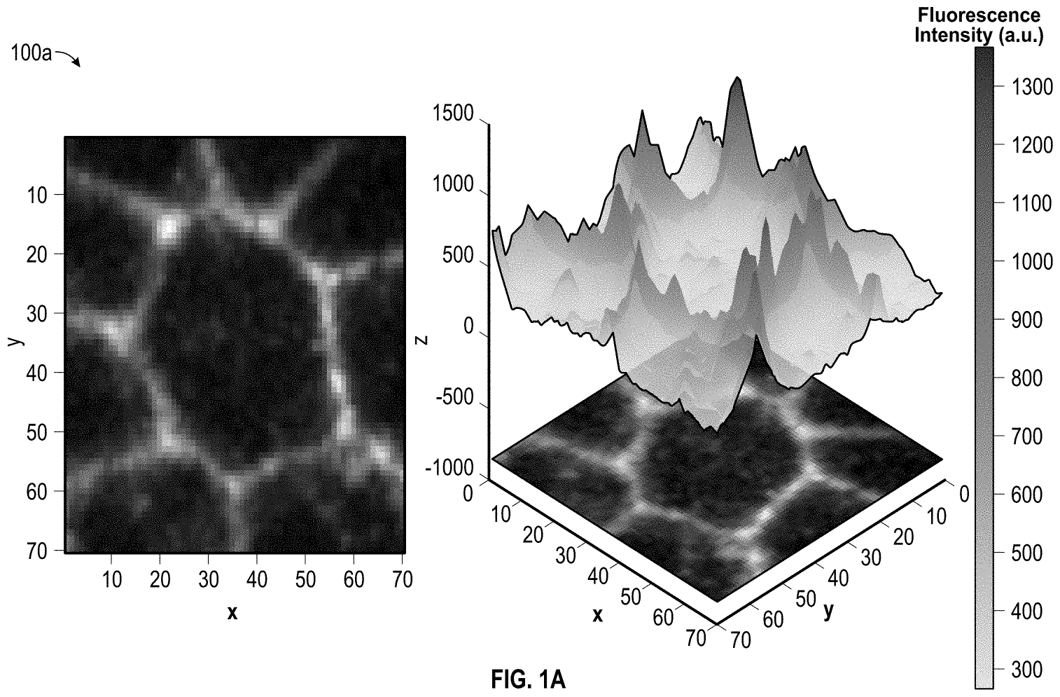

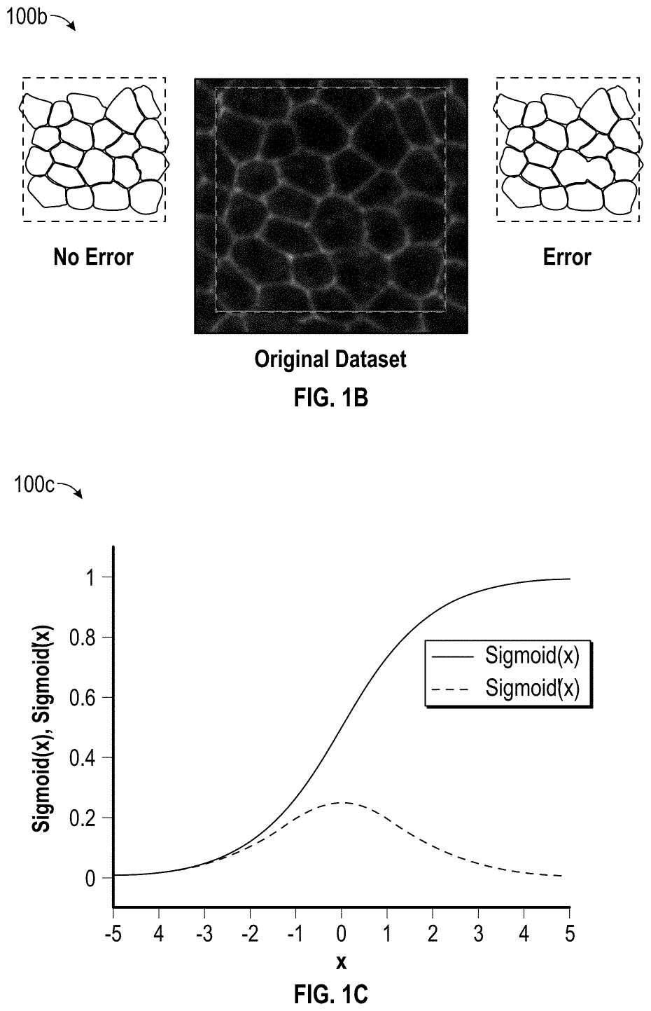

Referring to FIG. 1B is an observation image 100b depicting an error type image, in accordance with one or more exemplary embodiments. The observation image 100b includes a frequency observed error, in which multiple cells are detected as one cell.

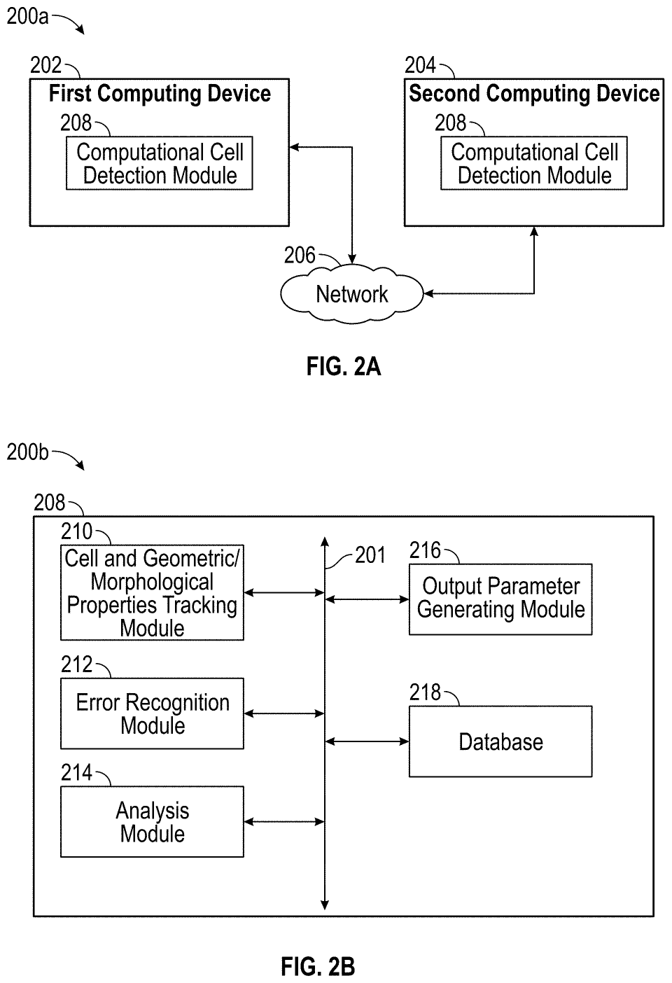

Referring to FIG. 1C is another observation image 100c depicting a graphical representation of sigmoid (x) and sigmoid′(x) of an action function, in accordance with one or more exemplary embodiments. The graphical representation of sigmoid (x) and sigmoid′(x) of an activation function illustrated in FIG. 1C. Sigmoid may define an activation function on a computing device 202 or 204 (refer FIG. 2A). The graph of sigmoid and sigmoid′ just represents how the values of the derivative change with respect to the function.

The sigmoid may define an activation function on the computing device 202 or 204 (refer FIG. 2A). The activation function receives an input of the weighted sum of the measurements of the geometric and morphological properties of cells and returns a value between 0 and 1, representing the ultimate output and prediction of the computational cell detection module 208 (refer FIG. 2A, FIG. 2B). In the case of the recognition of an error, the computational cell detection module 208 (refer FIG. 2A, FIG. 2B) predicts a number closer to 1. When no error is recognized, the computational cell detection module 208 (refer FIG. 2A, FIG. 2B) predicts a number closer to 0. The sigmoid function is defined by the equation:

$$σ ( x ) = \frac{1}{1 + {e}^{- x}}$$

For any input x, sigmoid (x) σ(C) always lies between 0 and 1. The derivative of sigmoid, σ′(x), describes the rate of change of σ(x) with respect to x. As x tends away from 0 in the positive and negative directions, the value of σ′(x) decreases as shown in FIG. 1b. σ′(x) may be written in terms of σ(x):

σ′(x)−σ(x)·[1−σ(x)]

This relationship between sigmoid, σ(x), and its derivative, σ′(x), is derived in (Eq. A.1). Written in terms of σ(x) as in above equation, σ′(x) may be efficiently calculated computationally without needing to compute all the steps derived in (Eq. A.1).

The relationship between sigmoid and its derivative is derived as follows (Eq. A.1):

$$σ ( x ) = \frac{1}{1 + {e}^{- x}}$$

$$\begin{matrix}{σ}^{′} ( x ) = \frac{[ ( 1 + {e}^{- x} ) \cdot ( 0 ) ] - [ ( 1 ) \cdot ( - 1 \cdot {e}^{- x} ) ]}{{( 1 + {e}^{- x} )}^{2}} \\ = \frac{{e}^{- x}}{{( 1 + {e}^{- x} )}^{2}} \\ = [ \frac{1}{1 + {e}^{- x}} ] \cdot [ \frac{1 + {e}^{- x} - 1}{1 + {e}^{- x}} ] \\ = [ \frac{1}{1 + {e}^{- z}} ] \cdot [ ( \frac{1 + {e}^{- x}}{1 + {e}^{- x}} ) - ( \frac{1}{1 + {e}^{- x}} ) ]\end{matrix} \\ ∴ {σ}^{′} ( x ) = σ ( x ) \cdot [ 1 - σ ( x ) ]$$

Referring to FIG. 2A is a block diagram 200a depicting a schematic representation of a system for the recognition of errors in the computational detection of cells in tissues from images based on input measurements about the geometric and morphological properties of the cells, in accordance with one or more exemplary embodiments. The images and cells may include, but are not limited to, cancerous cells, non-cancerous cells, malignant cells, carcinoma cells, sample cells, and the like. The system 200a includes a first computing device 202, a second computing device 204, and a network 206. The first computing device 202, and the second computing device 204 may be connected each other via the network 206. The network 206 may include but not limited to, a WIFI communication network e.g., the wireless high speed internet, or a combination of networks, a cellular service such as a 4G (e.g., LTE, mobile WiMAX) or 5G cellular data service, a RFID module, a NFC module, wired cables, such as the world-wide-web based Internet, or other types of networks may include Transport Control Protocol/Internet Protocol (TCP/IP) or device addresses (e.g. network-based MAC addresses, or those provided in a proprietary networking protocol, such as Modbus TCP, or by using appropriate data feeds to obtain data from various web services, including retrieving XML data from an HTTP address, then traversing the XML for a particular node), an Internet of things (IoT network devices), an Ethernet, a wireless local area network (WLAN), or a wide area network (WAN), and so forth without limiting the scope of the present disclosure. The network 206 may be configured to provide access to different types of users. The first computing device 202 and the second computing device 204 may include, but are not limited to, smart phones, personal computers, personal digital assistants, mobile stations, computing tablets, handheld devices, internet enabled calling devices, an internet enabled calling software, mobile phones, digital processing systems, and the like.

Although the first and second computing devices 202, 204 are shown in FIG. 1, an embodiment of the system 200a may support any number of computing devices. The first computing device 202 may be operated by a first user, and the second computing device 204 may be operated by a second user. The first user and the second may include, but are not limited to: scientists, researchers, doctors, medical practitioners, healthcare professionals, technicians, diagnosticians, laboratory research assistants, teaching staff, students, employees, owners, clients, and the like. The computing devices 202, 204 supported by the system 200a is realized as a computer-implemented or computer-based device having the hardware or firmware, software, and/or processing logic needed to carry out the computer-implemented methodologies described in more detail herein. The first computing device 202, and the second computing device 204, may include a computational cell detection module 208. The computational cell detection module 208, which is accessed as a mobile application, web application, software that offers the functionality of accessing mobile applications, and viewing/processing of interactive pages, for example, are implemented in the computing devices 202, 204 as will be apparent to one skilled in the relevant arts by reading the disclosure provided herein. For example, the computational cell detection module 208 may be any suitable application downloaded from GOOGLE PLAY® (for Google Android devices), Apple Inc.'s APP STORE® (for Apple devices), or any other suitable database, server, webpage or uniform resource locator (URL). The computational cell detection module 208 which may be a desktop application which runs on Mac OS, Microsoft Windows, Linux or any other operating system, and may be downloaded from a webpage or a CD/USB stick etc. In some embodiments, the computational cell detection module 208 may be software, firmware, or hardware that is integrated into the computing devices 202, 204. The computing devices 202, 204, may present a webpage to the user by way of a browser, wherein the webpage comprises a hyper-link may direct the user to uniform resource locator (URL).

The computational cell detection module 208 may be configured to allow the first and second users to share their identity credentials on the first and second computing devices 202, 204. The identity credentials may include, but are not limited to, mobile number, email identity, personal address details, license number, and so forth. The computational cell detection module 208 may be configured to predict and recognize errors based on input measurements of the geometric and morphological properties of cells. The computational cell detection module 208 may be configured to generate an output analysis on the computing device, the output analysis includes statically separable geometric and morphological properties of detected cells.

Referring to FIG. 2B is an example diagram 200b depicting the computational cell detection module 208 shown in FIG. 2A, in accordance with one or more exemplary embodiments. The computational cell detection module 208 may be configured to recognize errors in the detection of cells using information about geometric and morphological properties of cells chosen on the basis of statistical analyses. The computational cell detection module 208 includes a bus 201, a cell and geometric/morphological properties tracking module 210, an error recognition module 212, an analysis module 214, an output parameter generating module 216, and a database 218. The bus 201 may include a path that permits communication among the modules of the computational cell detection module 208 installed on the computing device 202 or 204. The term “module” is used broadly herein and refers generally to a program resident in the memory of the computing device 202 or 204.

The cell and geometric/morphological properties tracking module 210 may be configured to computationally identify and track individual cells within the living tissues and calculate information about the geometric and morphological properties of cells and shapes on the computing device 202 or 204. The error recognition module 212 may be configured to recognize errors based on input measurements of the geometric and morphological properties of the cells. The input measurements may include, but are not limited to, an area measurement, a perimeter measurement, an orientation measurement, an equivalent diameter measurement, and the like. In the case that an error is recognized, the error recognition module 212 predicts a number closer to 1 on the computing device 202 or 204. In case no error is recognized, the error recognition module 212 predicts a number closer to 0 on the computing device 202 or 204. The error recognition module 212 may include a target determining module 212a, and a prediction making module 212b (not shown). The target determining module 212a may be configured to determine a target value based on the input measurement. To compute the change backwards to update the weights and bias terms (before learning begins, these are initialized by a pseudorandom number generator), the change in cost of prediction c with respect to the change in prediction p is a partial derivative. The prediction making module 212b may be configured to make the weighted sum of the input measurements and the bias term.

The analysis module 214 may be configured to perform statistical analysis to identify geometric and morphological properties of cells and show statistically separable geometric and morphological properties of detected cells. The analysis module 214 may also be configured to generate measurements of the cells' geometric and morphological properties from the watershed-based cell detection on the computing device 202 or 204. The statistically separatable geometric and morphological properties may include, but are not limited to, an area property, a perimeter property, an orientation property, an equivalent diameter property, and the like. The output parameter generating module 216 may be configured to provide an effect of varying the learning rate, a, over different number of epochs. The output parameter generating module 216 may be configured to provide the measurements with information about erroneously detected cells on the computing device 202 or 204. The database 218 may be configured to record errors such as under-detection errors from the error recognition module 212.

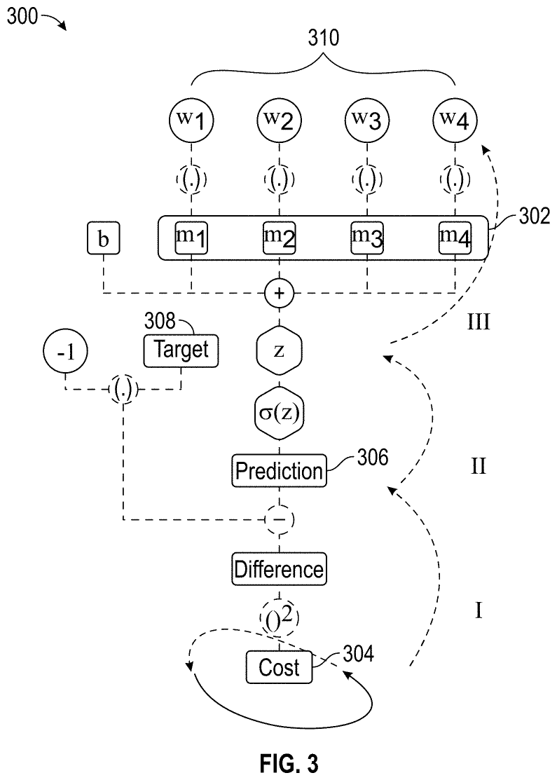

Referring to FIG. 3 is an example diagram 300 depicting an error-recognition information flow, in accordance with one or more exemplary embodiments. The error-recognition information flow 300 includes similarity to pattern recognition models that are basis for neural networks used in artificial intelligence algorithms. The error-recognition information flow 300 includes a list of relevant parameters:

m=Input measurement

w=Weight

b=Bias

[symbol] =Sum of weighted m and b

σ(z)=Activation function, sigmoid

σ′(z)=Derivative of sigmoid

p=Prediction of the model

c=Cost of prediction

α=Learning rate

The computational cell detection module 208 may be configured to recognize errors based on the input measurements m₁, m₂, m₃, m₄ 302. The input measurements 302 include an area measurement, a perimeter measurement, an orientation measurement, and an equivalent diameter measurement. The error-recognition information flow 300 includes a squared error cost function 304, a prediction 306, and a target 308. The squared error cost function 304 may define the cost, c, of the computational cell detection module 208's prediction, p 306, determined by the target value, t 308, based on the input measurement, mₙ:

c=(p−t)²

The target 308, t, may be defined by ground truth measurements which remain unchanged. To compute the change backwards and update the weights w1, w2, w3, w4 310 and bias terms, the change in c with respect to the change in p is the partial derivative, shown in step 1 in FIG. 3:

$$\frac{\partial c}{\partial p} = 2 \cdot p$$

To make the prediction, p, 306, the computational cell detection module 208 first sums the weighted sum of measurements and the bias term, b, which is the only term in the prediction that does not receive an input measurement. This yields an intermediary number, [symbol] :

[symbol]

=Σ(wₙ·mₙ)+b

These partial derivatives describe how [symbol] changes with respect to any weight, wₙ, or the bias term, b, shown in step III in FIG. 3:

$$\frac{\partial z}{\partial {w}_{n}} = {m}_{n} \\ \frac{\partial z}{\partial b} = 1$$

To arrive at p, [symbol] is input to sigmoid:

p=σ( [symbol] )

The rate of change of p with respect to the change in [symbol] is the derivative of sigmoid, described in the equation above and shown in step II in FIG. 3:

$$\frac{\partial p}{\partial z} = {σ}^{′} ( z ) = σ ( z ) \cdot [ 1 - σ ( z ) ]$$

The rates of changes in these intermediary values can be used to calculate the rate of change of the cost, c, with respect to any weight, wₙ, or bias, b:

$$\frac{\partial c}{\partial {w}_{n}} = \frac{\partial c}{\partial p} \cdot \frac{\partial p}{\partial z} \cdot \frac{\partial z}{\partial {w}_{n}}$$

$$\frac{\partial c}{\partial b} = \frac{\partial c}{\partial p} \cdot \frac{\partial p}{\partial z} \cdot \frac{\partial z}{\partial b}$$

Substituting the equations derived in above equations:

$$\frac{\partial c}{\partial {w}_{n}} = ( 2 \cdot p ) \cdot [ σ ( x ) \cdot ( 1 - σ ( x ) ) ] \cdot {m}_{n}$$

$$\frac{\partial c}{\partial b} = ( 2 \cdot p ) \cdot [ σ ( x ) \cdot ( 1 - σ ( x ) ) ] \cdot 1$$

The learning rate, α, is applied to update weight and bias terms:

$${w}_{updated} = {w}_{initial} - ( α \cdot \frac{\partial c}{\partial {w}_{initial}} )$$

$${b}_{updated} = {b}_{initial} - ( α \cdot \frac{\partial c}{\partial {b}_{initial}} )$$

The updated values of w and b may be substituted in the next prediction made by the computational cell detection module 208.

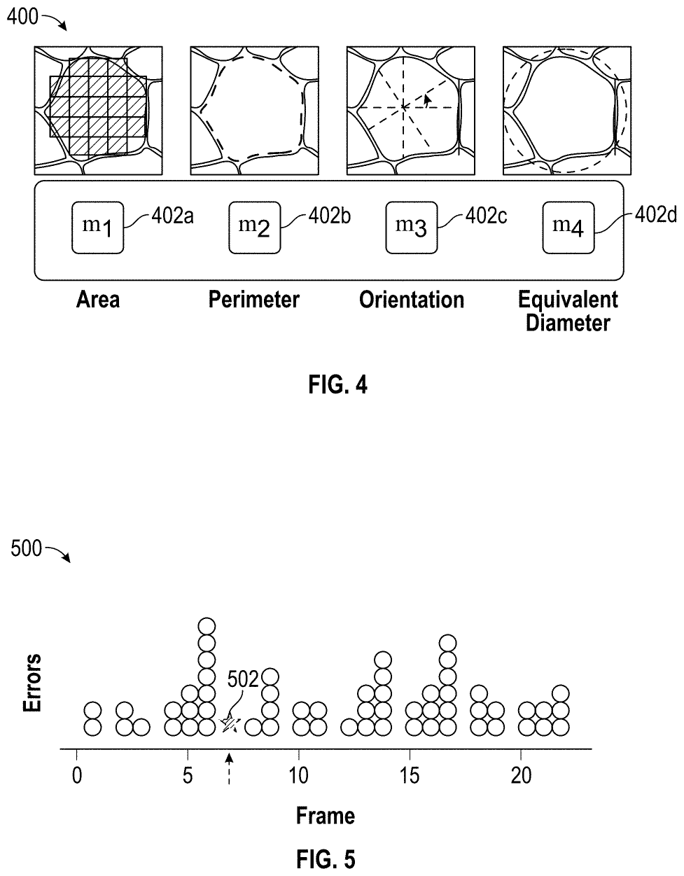

Referring to FIG. 4 is an example diagram 400 depicting diagrammatic representation of input measurements, in accordance with one or more exemplary embodiments. The diagrammatic representation of input measurements 400 includes m₁ 402a, m₂ 402b, m₃ 402c, and m₄ 402d. The input measurement, m₁ 402a, may be an area, the input measurement, m₂ 402b, may be a perimeter, the input measurement, m₃ 402c, may be an orientation, and the input measurement, m₄ 402d, may be an equivalent diameter.

Referring FIG. 5 is an example diagram 500 depicting frequency of occurrence of under-detection errors in an example dataset, in accordance with one or more exemplary embodiments. When the watershed algorithm was implemented on 22 separate images of the same population of 200 cells, 21 out of 22 images contained an under-detection error, while a single image 502 did not contain such an error. The single image 502 is marked with an arrow and a star in FIG. 5. The FIG. 500 is a dot plot of the number of under-detection errors in a sample dataset. The sample dataset includes ˜208 cells imaged 22 times on the microscope. The x axis represents frame number (1-22) and each dot on they axis represents one count of an under-detection error observed in that frame. In the frame marked by 502, there were no under-detection errors. FIG. 5 shows that this error occurs frequently (only one frame has no errors), even when the watershed algorithm is configured to detect cells to the best of its ability.

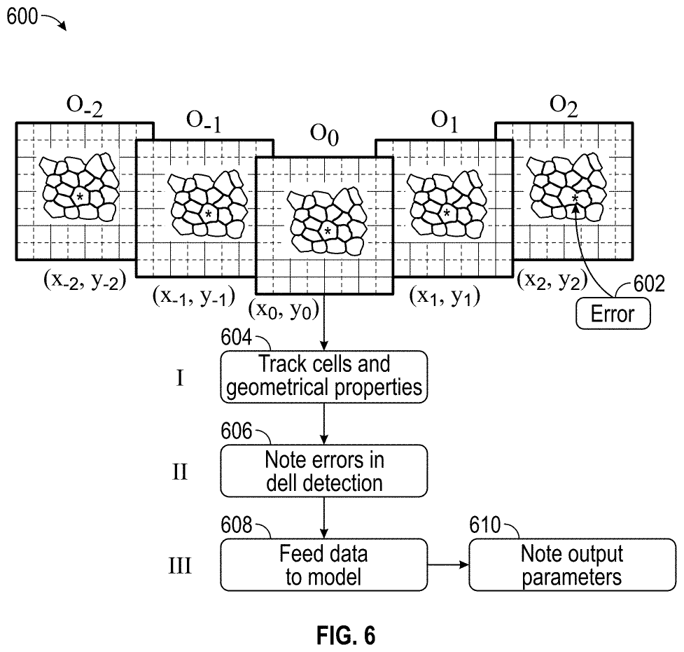

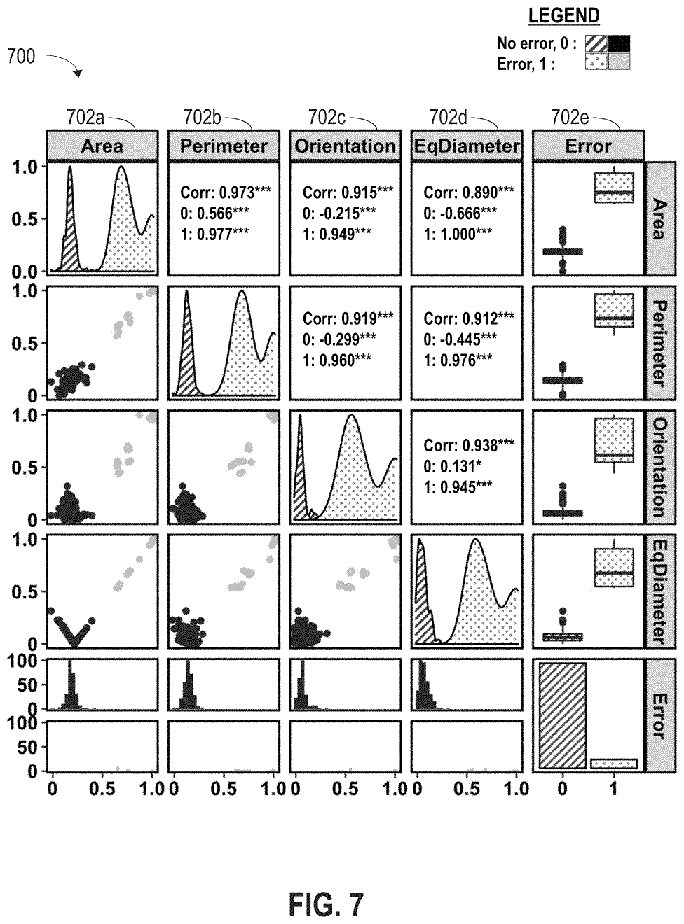

Referring to FIG. 6 is an example flow diagram 600 depicting a process by which information is collected, curated for ground truth, and fed to the computational cell detection module 208 to learn to recognize errors in the computational detection of cells within tissues, in accordance with one or more exemplary embodiments. The process 600 occurs through a series of successive iterations of updates (called epochs) to w and b based on input measurements. Performed in the sequence described in the diagram in FIG. 3, the process 600 includes input measurements with information about erroneously detected cells 602. An error may be observed (a ground truth error, meaning a human and not a computing device has identified it). At block 604, the computational cell detection module 208 may be configured to track detected cells and their geometric and morphological properties by minimizing the Euclidean distance of a cell's centroid from one frame to the next in order to locate the same cell. The geometric and morphological properties may be recorded. At block 606, the computational cell detection module 208 may be configured to record errors in cell detection. The errors that are observed as ground truth are recorded, the number 1 represents the case where an error is observed, and 0 is the case where it was not and then at block 608, the computational cell detection module 208 feeds the data to a computational model. In the arrow between 608 and 610, the computational model learns to recognize an error by looking at multiple measurements and iteratively updating w and b, which are initialized by a pseudorandom number generating algorithm. At block 610, the computational cell detection module 208 saves the output parameters after the learning process. These calculations constitute a computational model that may learn to recognize and predict errors made by the computational cell detection module 208 in the detection of cells, using information about the cells' geometric and morphological properties chosen on the basis of the statistical analyses in FIG. 7.

Referring to FIG. 7 is an example graph 700 depicting statistically separable geometric and morphological properties of cells, in accordance with one or more exemplary embodiments. The analysis of the geometric and morphological properties of cells 700 includes an area property 702a, a perimeter property 702b, an orientation property 702c, an equivalent diameter property 702d, and an erroneous detection property 702e. The example graph 700 represents the ground truth data (n=286 observations: 13 cells observed 22 times), grouped by the error or no error case of each observation. The dark color represents the no error case (0) and the light gray represents the error case (1). The example graph 700 demonstrates that when four specific properties are chosen, (viz. the area property 702a, the perimeter property 702b, the orientation property 702c, the equivalent diameter property 702d) and grouped by the error or no error status of each observation, the properties may be statistically separable according to the error or no error status (a computing device or human may draw lines between the error or no error cases in each panel). Statistical separability makes these specific geometric and morphological measurements apt choices for weights to apply to the measurements in the model in order to enable it to learn to linearly classify detection errors when it is shown multiple measurements of the same cell. The panels with text represent Pearson's correlation coefficient values for each group indicated by black (overall) dark gray, (error case) dark gray, and light gray (no error case). The erroneous detection property 702e includes erroneous cells.

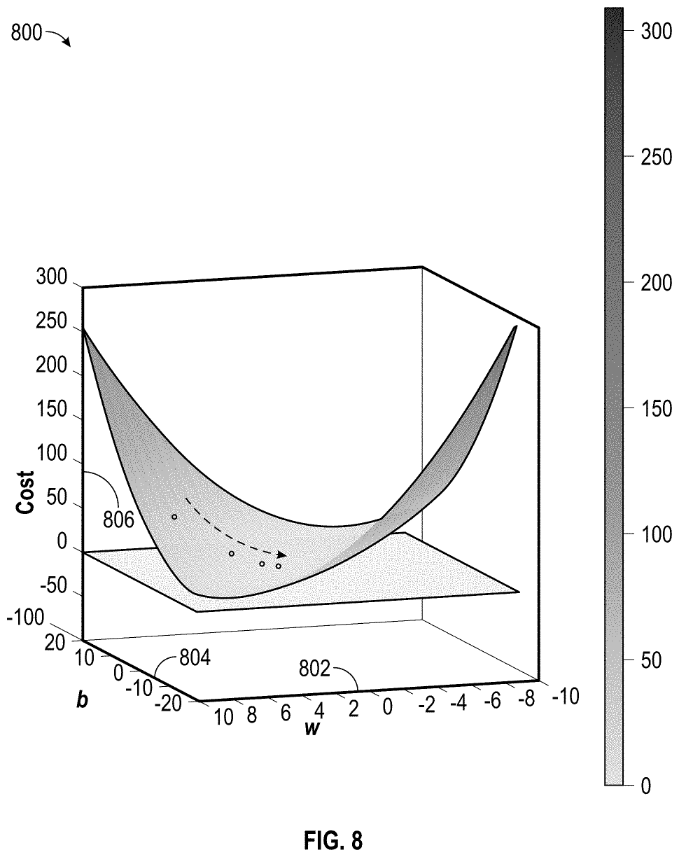

Referring to FIG. 8 is an example graph 800 depicting a graphical representation of the effect of updating weight w and bias b on the value of cost of prediction, in accordance with one or more exemplary embodiments. The graphical representation 800 describes the effect of four successive updates to any weight, wₙ, 802 and the bias, b, 804 on the cost, c, of prediction 806 (shown by an arrow and four dots tending towards the minimum). The three-axis graphical representation 800 depicting the effect of updating weights 802, bias 804 on the value of the cost 806. At the 0-value marker on the cost 806 axis. The effect of four successive updates may be shown by a dashed arrow. With every successive update, the value of the cost tends closer towards the minima Higher values of cost 806 are represented by darker shades of gray, and lower values are represented by lighted shades.

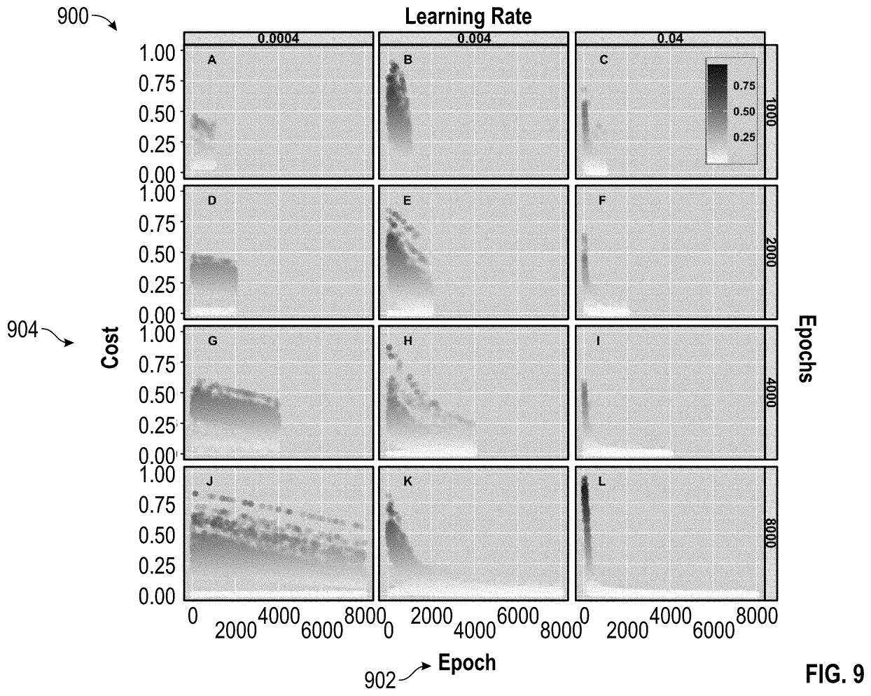

Referring to FIG. 9 is an example graph 900 depicting a pictorial representation of faceted graphs with each vertical panel showing the effect of varying learning rate, and each horizontal panel showing the effect of varying the number of epochs used by the computational cell detection module 208 to learn, in accordance with one or more exemplary embodiments. The pictorial representation 900 includes epoch values 902, and cost values 904. The computational cell detection module 208 is created such that the cost, c, 902 decreases over successive epochs 904, as the computational cell detection module 208 becomes progressively more successful at correctly recognizing errors. The series of successive iterations of updates to w and b based on input measurements are called epochs. The pictorial representation 900 further depicts the effect of varying the learning rate, a (the parameter that affects how much the computational cell detection module 208 is penalized for an incorrect prediction), over different numbers of epochs.

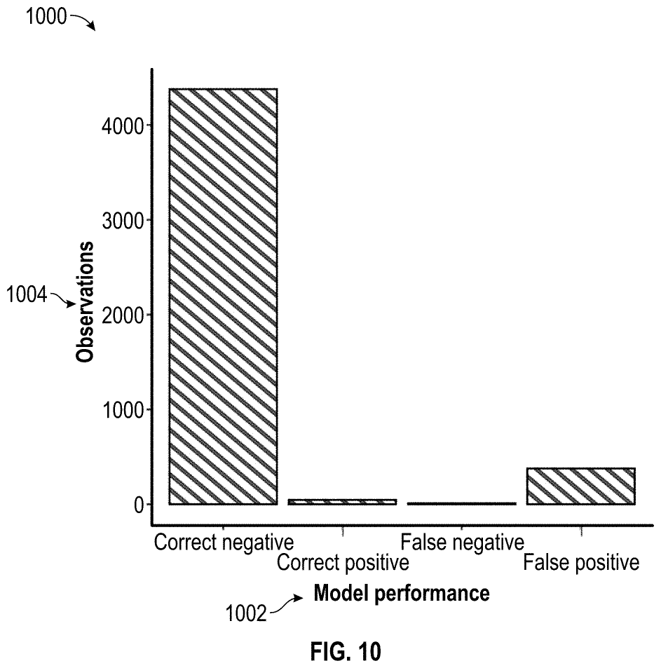

Referring to FIG. 10 is an example diagram 1000 depicting a performance model of the computational cell detection module 208 over the cells, in accordance with one or more exemplary embodiments. The performance model 1000 includes performance values 1002 and observations 1004. For example, the tested values of the computational cell detection module 208 on the observations 1004 of the cells. The observations 1004 may include 4576 observations of approximately 208 cells in each frame. The performance values 1002 may include correct negative, correct positive, false negative, false positive, and the like. The computational cell detection module 208 may be configured to correctly identify every single error appearing in every frame in all but one cell.

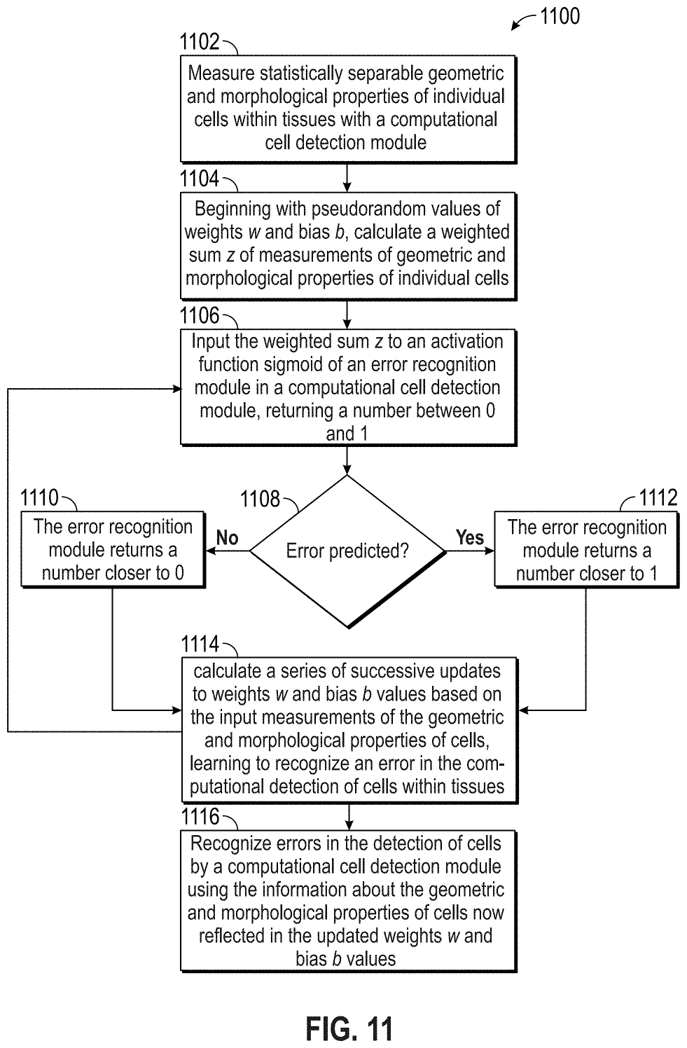

Referring to FIG. 11 is a flowchart 1100 depicting a method for the recognition of errors in the computational detection of cells from images based on input measurements of the geometric and morphological properties of cells, in accordance with one or more exemplary embodiments. The method 1100 may be carried out in the context of the details of FIG. 2A, FIG. 2B, FIG. 3, FIG. 4, FIG. 5, FIG. 6, FIG. 7, FIG. 8, FIG. 9, and FIG. 10. However, the method 1300 may also be carried out in any desired environment. Further, the aforementioned definitions may equally apply to the description below.

The method commences at step 1102, in which statistically separable geometric and morphological properties of individual cells within tissues are measured with a computational cell detection module on a computing device. Thereafter, at step 1104, beginning with pseudorandom values of weights and bias terms, a weighted sum of measurements of geometric and morphological properties of cells is calculated. Thereafter, at step 1106, the weighted sum is input to an activation function sigmoid of an error recognition module in a computational cell detection module on a computing device, returning a number between 0 and 1. Thereafter, at step 1108, asking whether the error recognition module predicts an error. In the case where the error recognition module predicts no error, at step 1110, the error recognition module returns a number closer to 0. In the case where the error recognition module predicts an error, at step 1112, the error recognition module returns a number closer to 1. Thereafter, at step 1114, the cost of the prediction is optimized by comparing against ground truth measurements of known errors, and a series of successive updates to weights and bias values is calculated based on the input measurements of the geometric and morphological properties of cells, comprising a step by which the model learns to recognize an error in the computational detection of cells within tissues. Thereafter, at step 1116, the updated values of weights and bias that are recorded by the trained model are used to predict and recognize errors in the computational detection of cells in any dataset by any computing device.

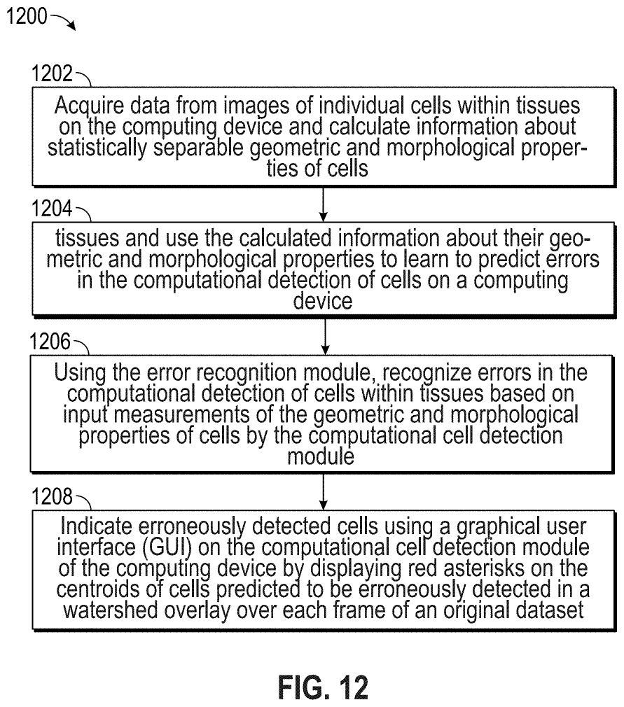

Referring to FIG. 12 is a flowchart 1200 depicting a method for indicating erroneous cells using a GUI, in accordance with one or more exemplary embodiments. The method 1200 may be carried out in the context of the details of FIG. 2A, FIG. 2B, FIG. 3, FIG. 4, FIG. 5, FIG. 6, FIG. 7, FIG. 8, FIG. 9, FIG. 10, and FIG. 11. However, the method 1200 may also be implemented, incorporated, or carried out in any desired environment, interface, algorithm or analysis workflow. Further, the aforementioned definitions may equally apply to the description below.

The method commences at step 1202, in which data from images of individual cells within the tissues is acquired on the computing device by the computational cell detection module and information about the geometric and morphological properties of individual cells is calculated. Thereafter, at step 1204, computationally detect and track individual cells within tissues and use the information about the geometric and morphological properties of cells with the computational cell detection module on the computing device and use the information about the geometric and morphological properties to learn to predict and recognize errors in the computational detection of cells. Thereafter, at step 1206, use the error recognition module to predict and recognize errors in the computational detection of cells within tissues based on input measurements of the geometric and morphological properties of cells. Thereafter, at step 1208, indicate erroneously detected cells using a GUI on the computational cell detection module of the computing device by displaying a red asterisk on the centroids of erroneously detected cells in a watershed overlay over each frame of an original dataset.

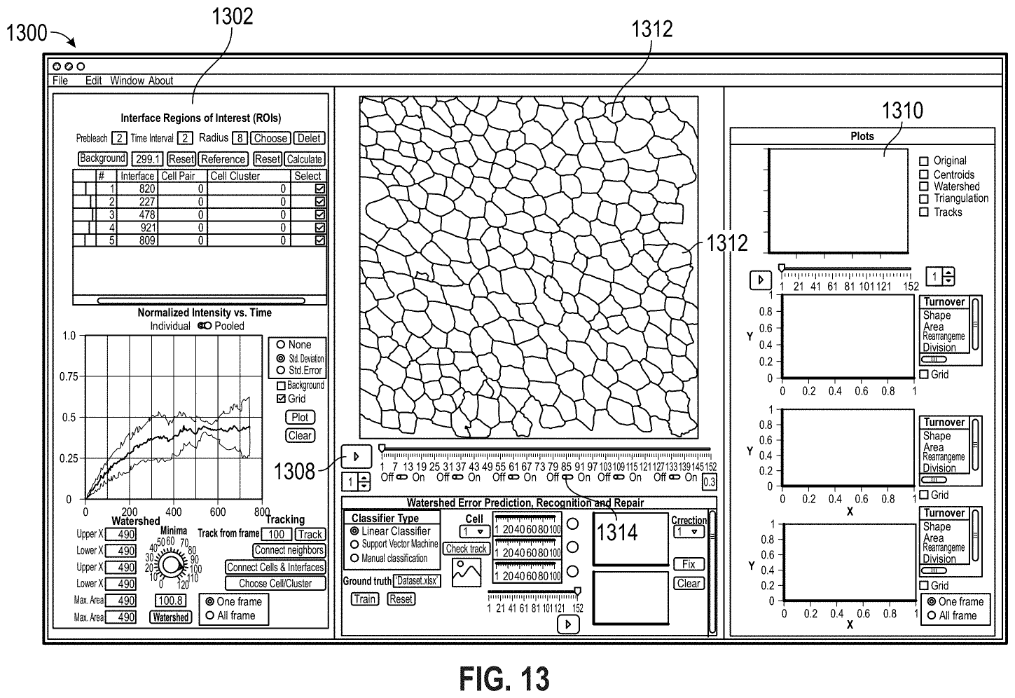

Referring to FIG. 13 is an example diagram 1300 depicting a GUI, in accordance with one or more exemplary embodiments. The GUI 1300 includes a panel for subcellular scale dynamics analysis 1302, a normalized fluorescence intensity over time graph 1304, a table of regions of interest selected in a dataset 1306, a playback option 1308, plot spaces 1310, and a display unit 1312. Datasets may be loaded via a file menu bar. Prompts enable the user to load timelapses containing information about photobleached experiments. Upon loading the dataset, the computational cell detection module 208 automatically calculates the number of frames and the display unit 1312 is configured to show the number of frames. The user may scroll through the playback option 1308 or press play to display each frame of the dataset or selected overlay (by slider switches 1314) on the display unit 1312. The panel for subcellular scale dynamics analysis 1302 includes a table showing the interface regions of interest selected between cells 1306. The panel 1302 includes buttons configured to calculate the background, reference, and region of interest fluorescence intensities using interactive input from the user to indicate, either by tracing or clicking, the regions of a time-lapse dataset containing these respective intensities.

In accordance with one or more exemplary embodiments of the present disclosure, the panel for subcellular scale dynamics analysis 1302 may also include options configured to provide a numerical input via a keyboard to indicate the number of pre-bleach frames and radius of a circular interface region of interest and these options configured to select and delete certain interfaces after selection also exist. In the case of a dataset containing no time-lapse or photobleach data, the options not to use the panel 1302 or to inactivate its interactivity may be available. The calculate buttons may be configured to run a series of calculations for each interface to double-normalize (against background and against reference pre- and post-bleach intensities) and report the value of the double-normalized fluorescence intensity over time.

In accordance with one or more exemplary embodiments of the present disclosure, the normalized fluorescence intensity over time graph 1304 may be configured to show the double-normalized fluorescence intensity over time. The option exists to monitor the standard deviation and standard error of the selected interfaces. Furthermore, the option also exists to view each interface separately or pool them all together and see the net result via a toggle switch selecting between “Individual” and “Pooled” graphs. To clear the graph, the user clicks “clear”. Selecting different combinations of interfaces by checkbox in the table of regions of interest 1306 shows the calculations for that particular set of interfaces in the tissue and automatically updates the plot space 1304 to reflect the selected. The option exists to either display or not display a grid on the plot space. The option exists to save a plot as a PNG or JPG file. An option may be configured to export the normalized intensity values as a spreadsheet. The slider switch 1314 may be configured to enable the user to choose to display the selected interfaces on the display unit 1312. Together with the results of the watershed-based cell detection in panel 1302, the subcellular-scale dynamics analysis results are displayed in the plot spaces 1310 alongside the cell- and tissue-scale dynamics analysis results.

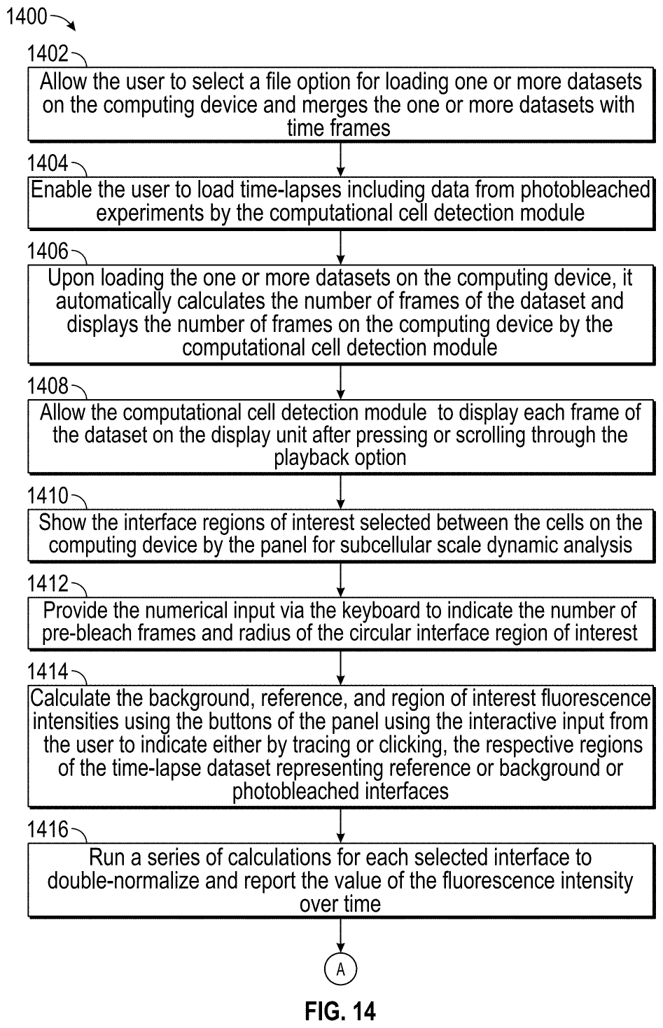

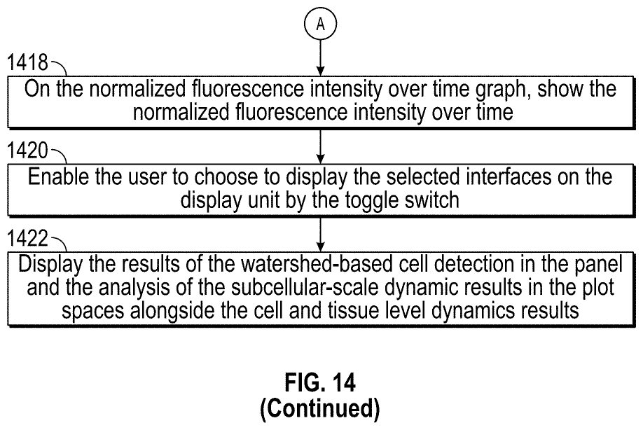

Referring to FIG. 14 is a flowchart 1400 depicting a method for displaying watershed-based cell detection and the analysis of the subcellular-scale dynamics results by using the GUI, in accordance with one or more exemplary embodiments. The method 1400 may be carried out in the context of the details of FIG. 2A, FIG. 2B, FIG. 3, FIG. 4, FIG. 5, FIG. 6, FIG. 7, FIG. 8, FIG. 9, FIG. 10, FIG. 11, FIG. 12, and FIG. 13. However, the method 1400 may also be implemented, incorporated, or carried out in any desired environment, interface, algorithm or analysis workflow. Further, the aforementioned definitions may equally apply to the description below.

The method commences at step 1402, in which the GUI allows the user to select the file option to load one or more datasets. Thereafter, at step 1404, the GUI determines whether the dataset contains time-lapse information. If yes, proceed to step 1406, in which the GUI counts and displays the time-lapse frames enabling playback scrolling interactivity by the user. If the answer is “no” at step 1404, and time-lapse information is not detected, then proceed to 1432, enabling the interactivity of the computational cell detection module and the error recognition module. After step 1406, proceed to step 1408, in which the GUI enables the cell tracking option. Thereafter, at step 1410, determine whether the dataset includes photobleaching information. If yes, at step 1412, the GUI enables the subcellular scale dynamics analysis panel. If no, proceed to step 1432, in which the GUI enables the interactivity of the computational cell detection module and the error recognition module. From step 1412, in which the subcellular scale dynamics analysis panel is enabled, proceed to step 1414, in which the GUI receives numerical inputs from the user via the keyboard that indicate the number of pre-bleach frames and the radius of the circular regions of interest. Thereafter, at step 1416, calculate the background, reference, and region of interest fluorescence intensities using the buttons of the panel receiving interactive input from the user to indicate either by tracing or clicking, the respective regions of the time-lapse dataset representing reference or background or photobleached interfaces. Thereafter, at step 1418, list the interfaces in the subcellular scale dynamics panel and enable selection checkboxes; this information may also be used by the display unit of the GUI at step 1448, if the interfaces slider switch is set to “on” by the user, the selected interface regions of interest are displayed by a red circle over the original dataset. Thereafter, at step 1420, when the “Calculate” button is pressed by the user, run a series of calculations for each selected interface to double-normalize and report the value of the fluorescence intensity at each frame. Thereafter, at step 1422, show the normalized fluorescence intensity over time. Thereafter, at step 1424, if the toggle switch is set to “Pooled”, and the answer is yes, then at step 1428, pool the selected interfaces' normalized intensity and display the average normalized fluorescence intensity values at each frame, if selected by the user, also display either the standard error or the standard deviation of this measurement. At step 1424, if the toggle switch is not set to “Pooled”, then at step 1426 the toggle switch automatically flipped to “Individual”, and at step 1430, each interface's double normalized intensity values are graphed individually, and selecting and de-selecting an interface in the list retains and removes that interface respectively in the graph. If at 1426, the toggle switch is not set to “Individual,” then it is set to “Pooled”, and the GUI returns to step 1424 and step 1428. After step 1430, the GUI may be allowed to proceed by the user to step 1450, in which graphs of subcellular-scale, cell-scale, and tissue-scale dynamics analyses results are displayed alongside each other and data may be exported in a chosen format. At step 1410, if photobleaching information is not included in the dataset, the subcellular scale dynamics analysis panel is not enabled, and at step 1432, the computational cell detection module and the error recognition module are enabled. Then, at step 1434, using interactive inputs from the user via the keyboard that set the bounds of the field of view and fluorescence intensity threshold, the GUI runs the computational cell detection module and the error recognition module. Then, at step 1436, in case of an over-detection error in cell detection, the label matrix of detected cells is edited to replace false background pixels in the dataset with correct label numbers, and in the case of an under-detection error, user input via freehand trace is used to add missing background pixels and a new label for the additional cell or cells. Thereafter, at step 1438, the geometric and morphological properties of the corrected cells are re-calculated and neighboring cell centroids are connected. Thereafter, at step 1440, if cell tracking is not enabled, at step 1442, the user can choose to export either numerical or graphical data from cells of their selection or from all detected cells. If at step 1440, cell tracking is enabled, then proceed to step 1444, when the “Track Cells” button is pressed, track cells from specified frame received by numerical input via the keyboard from the user, and retaining colormap information from frame to frame. Thereafter, at step 1446, calculate the rates of change of geometric and morphological properties of each tracked cell and of the tissue represented by the cells, and save as track tables in a relational database. At step 1448, these track tables and geometric and morphological properties may be displayed on the display unit as per the user's choice of slider switch. After step 1446, the information may also be directly exported as numerical data in a relational database or as graphical data from plot spaces at step 1450.

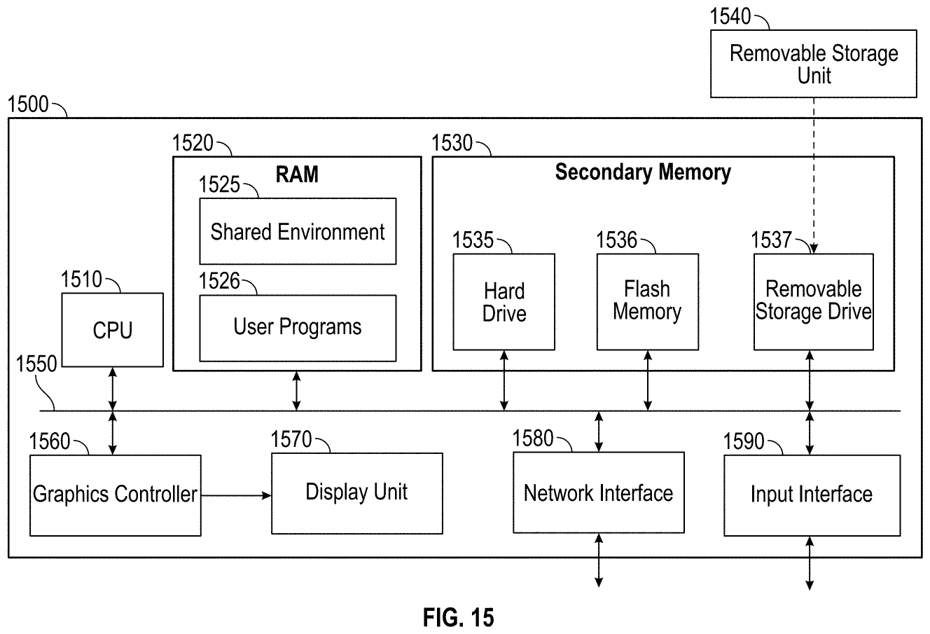

Referring to FIG. 15 is a block diagram 1500 illustrating the details of a digital processing system 1500 in which various aspects of the present disclosure are operative by execution of appropriate software instructions. The Digital processing system 1500 may correspond to the computing devices 202, 204 (or any other system in which the various features disclosed above can be implemented).

Digital processing system 1500 may contain one or more processors such as a central processing unit (CPU) 1510, random access memory (RAM) 1520, secondary memory 1530, graphics controller 1560, display unit 1570, network interface 1580, and input interface 1590. All the components except display unit 1570 may communicate with each other over communication path 1550, which may contain several buses as is well known in the relevant arts. The components of FIG. 15 are described below in further detail.

CPU 1510 may execute instructions stored in RAM 1520 to provide several features of the present disclosure. CPU 1510 may contain multiple processing units, with each processing unit potentially being designed for a specific task. Alternatively, CPU 1510 may contain only a single general-purpose processing unit.

RAM 1520 may receive instructions from secondary memory 1530 using communication path 1550. RAM 1520 is shown currently containing software instructions, such as those used in threads and stacks, constituting shared environment 1525 and/or user programs 1526. Shared environment 1525 includes operating systems, device drivers, virtual machines, etc., which provide a (common) run time environment for execution of user programs 1526.

Graphics controller 1560 generates display signals (e.g., in RGB format) to display unit 1570 based on data/instructions received from CPU 1510. Display unit 1570 contains a display screen to display the images defined by the display signals. Input interface 1590 may correspond to a keyboard and a pointing device (e.g., touchpad, mouse) and may be used to provide inputs. Network interface 1580 provides connectivity to a network (e.g., using Internet Protocol), and may be used to communicate with other systems (such as those shown in FIG. 2A, FIG. 2B) connected to the network 206.

Secondary memory 1530 may contain hard drive 1535, flash memory 1536, and removable storage drive 1537. Secondary memory 1530 may store the data software instructions (e.g., for performing the actions noted above with respect to the figures), which enable digital processing system 1500 to provide several features in accordance with the present disclosure.

Some or all of the data and instructions may be provided on removable storage unit 1540, and the data and instructions may be read and provided by removable storage drive 1537 to CPU 1510. Floppy drive, magnetic tape drive, CD-ROM drive, DVD Drive, Flash memory, removable memory chip (PCMCIA Card, EEPROM) are examples of such removable storage drive 1537.

Removable storage unit 1540 may be implemented using medium and storage format compatible with removable storage drive 1537 such that removable storage drive 1537 can read the data and instructions. Thus, removable storage unit 1540 includes a computer readable (storage) medium having stored therein computer software and/or data. However, the computer (or machine, in general) readable medium can be in other forms (e.g., non-removable, random access, etc.).

In this document, the term “computer program product” is used to generally refer to removable storage unit 1540 or hard disk installed in hard drive 1535. These computer program products are means for providing software to digital processing system 1500. CPU 1510 may retrieve the software instructions, and execute the instructions to provide various features of the present disclosure described above.

The term “storage media/medium” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage memory 1530. Volatile media includes dynamic memory, such as RAM 1520. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus (communication path) 1550. Transmission media can also take the form of acoustic or light waves, such as those generated during radio-wave and infra-red data communications.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Furthermore, the described features, structures, or characteristics of the disclosure may be combined in any suitable manner in one or more embodiments. In the above description, numerous specific details are provided such as examples of programming, software modules, user selections, network transactions, database queries, database structures, hardware modules, hardware circuits, hardware chips, etc., to provide a thorough understanding of embodiments of the disclosure.

Although the present disclosure has been described in terms of certain preferred embodiments and illustrations thereof, other embodiments and modifications to preferred embodiments may be possible that are within the principles and spirit of the system and method in this description. The above descriptions and figures are therefore to be regarded as illustrative and not restrictive.

Thus the scope of the present disclosure is defined by the appended claims and includes both combinations and sub-combinations of the various features described here in above as well as variations and modifications thereof, which might occur to any persons skilled in the art upon reading the foregoing description.

What is claimed is:

1. A system that can learn to recognize and predict one or more errors in computational detection of cells within tissues from one or more images based on one or more input measurements about one or more geometric and morphological properties of one or more cells, and perform tissue-, cell-, and subcellular-scale dynamics analyses within a single user interface, comprising:
a computational cell detection module configured to take the one or more input measurements and computationally identify one or more cells within tissues and calculate information about the one or more geometric and morphological properties of the one or more cells on a computing device, whereby the one or more input measurements comprising at least one of: an area measurement; a perimeter measurement; an orientation measurement; and an equivalent diameter measurement; the computational cell detection module configured to implement a single-layered neural network for detecting a plurality of abnormal cell shapes within the tissues, the one or more geometric and morphological properties comprising at least one of: an area property; a perimeter property; an orientation property; and an equivalent diameter property; the computational cell detection module comprising a graphical user interface (GUI) configured to display and enable the user to export the results of accurate watershed-based cell detection and to display and enable the user to export an analysis of subcellular-scale dynamics results alongside cell- and tissue-scale dynamics analyses results calculated from cell detection on the computing device, the computational cell detection module configured to generate measurements of the one or more cells' the one or more geometric and morphological properties from the watershed-based cell detection on the computing device and the graphical user interface (GUI) configured to display interface regions of interest selected between the one or more cells on the computing device for the subcellular-scale dynamics results; and
the computational cell detection module comprising a sigmoid configured to take a weighted sum of measurements of the one or more geometric and morphological properties and bias and return a value between 0 and 1, the computational cell detection module configured to predict a number closer to 1 in the case of recognition of one or more errors and predict a number closer to 0 when no error is recognized; the computational cell detection module configured to predict and recognize the one or more errors such as under-detection errors within the tissues and record the one or more errors and feed the one or more errors to a database, the computational cell detection module configured to generate an output analysis on the computing device, the output analysis comprising statically separable geometric and morphological properties of detected cells.

2. The system of claim 1, wherein the computational cell detection module is configured to perform a series of successive iterations of updates to a plurality of parameters based on the one or more input measurements on the computing device.

3. The system of claim 2, wherein the plurality of parameters comprising the one or more input measurements, one or more weights, one or more bias, one or more sum of weighted measurement and bias, the sigmoid, a derivative of sigmoid, prediction, cost of prediction, and a learning rate.

4. The system of claim 1, wherein the computational cell detection module is configured to enable a user to train a computational model on datasets and experiment with varying the learning rate parameter to adapt the computational model to the datasets on the computing device.

5. The system of claim 1, wherein the computational cell detection module is configured to learn to predict and recognize the one or more errors in algorithmic detection of the one or more cells based on a plurality of observations of the one or more geometric and morphological properties of the one or more cells within the tissues.

6. The system of claim 1, wherein the single-layered neural network of the computational cell detection module is configured to achieve accurate results, more interpretable, adaptable, and accessible to the user who wants to feed the computational cell detection module a variety of information about the one or more geometric and morphological properties of the one or more cells within the tissues.

7. The system of claim 1, wherein the single-layered neural network of the computational cell detection module is configured to reduce overall computational time and effort with accurate results on the computing device.

8. The system of claim 1, wherein the computational cell detection module is configured to predict and recognize the one or more errors in the datasets based on the one or more input measurements of the one or more geometric and morphological properties of the one or more cells.

9. The system of claim 1, wherein the computational cell detection module is configured to acquire data from the one or more images of the one or more cells that are adhered to each other on the computing device.

10. The system of claim 1, wherein the computational cell detection module is configured to use the single-layered neural network, and scripts written without use of one or more libraries such as a TensorFlow.

11. A method for error prediction and recognition in computational detection of one or more cells from one or more images based on one or more input measurements, comprising:
inputting a plurality of parameters of one or more cells to a computational cell detection module on a computing device, the plurality of parameters comprising at least one of: the one or more input measurements; one or more weights; one or more bias; one or more sum of weighted measurement and bias; a sigmoid; a derivative of sigmoid; a prediction; a cost of prediction; and a learning rate;
inputting a weighted sum of the one or more input measurements of one or more geometric and morphological properties of the one or more cells to a sigmoid of an error recognition module within the computational cell detection module and returning a value between 0 and 1;
returning a number closer to 1 in the case of prediction and recognition of the one or more errors and return a number closer to 0 when no error is recognized by the computational cell detection module on the computing device;
performing a series of successive iterations of updates to the plurality of parameters based on the one or more input measurements on the computing device by the computational cell detection module; and
predicting and recognizing the one or more errors in the detection of cells on the computational cell detection module using information about the one or more geometric and morphological properties of the one or more cells chosen on a basis of a statistical analysis, the statistical analysis comprising statically separable geometric and morphological properties of detected cells, the computational cell detection module comprising a graphical user interface (GUI) configured to display and enable the user to export the results of accurate watershed-based cell detection and to display and enable the user to export an analysis of subcellular-scale dynamics results alongside cell- and tissue-scale dynamics analyses results calculated from cell detection on the computing device, the computational cell detection module configured to generate measurements of the one or more cells' the one or more geometric and morphological properties from the watershed-based cell detection on the computing device and the graphical user interface (GUI) configured to display interface regions of interest selected between the one or more cells on the computing device for the subcellular-scale dynamics results.

12. The method of claim 11, comprising a step of predicting and recognizing computational errors such as under-detection errors of cells within tissues and predicting and recognizing errors in datasets based on the one or more input measurements of the one or more geometric and morphological properties of the one or more cells by the computational cell detection module.

13. The method of claim 11, comprising a step of indicating one or more erroneous cells using the graphical user interface (GUI) by the computational cell detection module in each frame with a red asterisk on a centroid on the watershed overlay over the original dataset displayed on the computing device.

14. A computer program product comprising a non-transitory computer-readable medium having a computer-readable program code embodied therein to be executed by one or more processors, said program code including instructions to:
input a plurality of parameters of one or more cells to a computational cell detection module on a computing device, the plurality of parameters comprising at least one of: the one or more input measurements; one or more weights; one or more bias; one or more sum of weighted measurement and bias; a sigmoid; a derivative of sigmoid; a prediction; a cost of prediction; and a learning rate;
input a weighted sum of the one or more input measurements of one or more geometric and morphological properties of the one or more cells to a sigmoid of an error recognition module within the computational cell detection module and returning a value between 0 and 1;
return a number closer to 1 in the case of prediction and recognition of the one or more errors and return a number closer to 0 when no error is recognized by the computational cell detection module on the computing device;
perform a series of successive iterations of updates to the plurality of parameters based on the one or more input measurements on the computing device by the computational cell detection module; and
predict and recognize the one or more errors in the detection of cells on the computational cell detection module using information about the one or more geometric and morphological properties of the one or more cells chosen on a basis of a statistical analysis, the computational cell detection module comprising a graphical user interface (GUI) configured to display and enable the user to export the results of accurate watershed-based cell detection and to display and enable the user to export an analysis of subcellular-scale dynamics results alongside cell- and tissue-scale dynamics analyses results calculated from cell detection on the computing device, the computational cell detection module configured to generate measurements of the one or more cells' the one or more geometric and morphological properties from the watershed-based cell detection on the computing device and the graphical user interface (GUI) configured to display interface regions of interest selected between the one or more cells on the computing device for the subcellular-scale dynamics results.

15. The computer program product of claim 14, wherein the computational cell detection module comprising an error recognition module configured to recognize one or more errors based on the one or more input measurements of the one or more geometric and morphological properties of the one or more cells.

16. The computer program product of claim 14, wherein the computational cell detection module comprising an analysis module configured to perform the statistical analysis to identify the one or more geometric and morphological properties of the one or more cells and show statistically separable geometric and morphological properties of the one or more cells.

17. The computer program product of claim 14, wherein the computational cell detection module comprising an output parameter generating module configured to provide an effect of varying the plurality of parameters and provide the one or more input measurements with information about erroneously detected cells on the computing device.
